(19) United States
(12) Patent Application Publication
(10) Pub. No.: US 2024/0206817 A1
(43) Pub. Date: Jun. 27, 2024

(54) INTEGRATED SENSING PLATFORM

(71) Applicant: SAMSUNG ELECTRONICS CO., LTD., Suwon-si (KR)

(72) Inventors: Byung Hoon KO, Suwon-si (KR); Jae Min KANG, Suwon-si (KR); Sang Kon BAE, Suwon-si (KR)

(73) Assignee: SAMSUNG ELECTRONICS CO., LTD., Suwon-si (KR)

(21) Appl. No.: 18/124,824

(22) Filed: Mar. 22, 2023

(30) Foreign Application Priority Data
Dec. 27, 2022 (KR) 10-2022-0185960

(51) Int. Cl. A61B 5/00; A61B 5/01; A61B 5/0531; A61B 5/318

(52) U.S. Cl. CPC A61B 5/72; A61B 5/01; A61B 5/0531; A61B 5/318; A61B 2560/0223; A61B 2560/0252

(57) ABSTRACT

An integrated sensing platform is provided. The integrated sensing platform may include a first sensor configured to obtain a first signal from a measurement target; at least one calibration temperature sensor configured to obtain a temperature signal around the first sensor, and a circuit layer electrically connected to the first sensor and the calibration temperature sensor and configured to correct the first signal obtained from the first sensor on the basis of the temperature signal obtained from the calibration temperature sensor.

### CROSS-REFERENCE TO RELATED APPLICATION(S)

This application is based on and claims priority under 35 USC § 119(a) from Korean Patent Application No. 10-2022-0185960, filed on Dec. 27, 2022, in the Korean Intellectual Property Office, the entire disclosure of which is incorporated herein by reference for all purposes.

### BACKGROUND

### 1. Field

The disclosure relates to a sensing platform that acquires information on a measurement target.

### 2. Description of Related Art

With the aging population, increased medical costs, and a lack of medical personnel for specialized medical services, research is being conducted on information technology (IT)-medical convergence technologies, in which IT technology and medical technology are combined. For example, health monitoring systems are being developed, which provide extended care to patients away from the hospital. In particular, the health monitoring systems may obtain bio-signals of a patient (or a user) while the patient is away from the hospital (e.g., home or office) so that the health status of the patient can be monitored in daily life.

Some examples of bio-signals, which indicate the health condition of individuals, may include an electrocardiogramaignal, a photoplethysmogram (PPG) signal, an electromyography (EMG) signal, and the like, and various bio-signal sensors are being developed to measure the bio-signals in daily life.

For example, the PPG sensor may estimate biometric information, such as heart rate, oxygen saturation, blood pressure, etc. of a human body by analyzing a pulse waveform which reflects a condition of the cardiovascular system and the like. Thus, when biometric information is estimated using a PPG signal, a method for acquiring an accurate PPG signal is required in order to increase estimation accuracy.

### SUMMARY

According to an aspect of the disclosure, there is provided an integrated sensing platform including: a first sensor configured to obtain a first signal from a measurement target; at least one calibration temperature sensor configured to obtain a temperature signal in a vicinity of the first sensor; and a circuit layer electrically connected to the first sensor and the at least one calibration temperature sensor and comprising a processor configured to: obtain the first signal from the first sensor; obtain the temperature signal from the at least one calibration temperature sensor; and adjust the first signal based on the temperature signal.

The processor may be further configured to adjust the first signal by inputting first signal data obtained from the first sensor and temperature data obtained from the at least one calibration temperature sensor into a first signal correction model.

The first sensor may include a light source mounted on the circuit layer and configured to emit light to the measurement target and a first light receiver mounted on the circuit layer and configured to receive light reflected from the measurement target, wherein the integrated sensing platform may further include a sensor cover having a portion that allows the light to pass through to the measurement target.

The first sensor may include one of a photoplethysmogram (PPG) sensor or an optical displacement sensor.

The light source may be a multi-wavelength light source.

The integrated sensing platform may further include an optical splitter configured to distribute the light emitted by the light source to a measurement target contact area and a reflective area of the sensor cover; and a second light receiver mounted on the circuit layer and configured to receive light reflected from the reflective area and obtain a contact force signal of the measurement target with respect to the measurement target contact area, wherein the processor is further configured to adjust the first signal obtained from the first light receiver based on the contact force signal obtained from the second light receiver along with the temperature signal obtained from the at least one calibration temperature sensor.

The processor may be further configured to adjust the first signal by inputting first signal data obtained from the first light receiver, temperature data obtained from the at least one calibration temperature sensor, and contact force data obtained from the second light receiver into a first signal correction model.

The first sensor may be an optical force sensor that includes a light source mounted on the circuit layer to emit light to a reflective area of a sensor cover pressed by a force of the measurement target and a first light receiver mounted on the circuit layer to receive light reflected from the reflective area of the sensor cover.

The first sensor may include a light receiver mounted on the circuit layer and configured to receive light radiated from the measurement target, and wherein the integrated sensing platform further comprises a sensor cover having a portion that allows the light to pass from to the measurement target.

The first sensor may include an optical temperature sensor or a light intensity sensor.

The first sensor may include a sensing electrode that is electrically connected to the circuit layer, and wherein the first sensor is provided on an outer surface of a sensor cover covering the circuit layer.

The first sensor may be an electrocardiogram (ECG) sensor configured to acquire an electrocardiogram signal of a human body in contact with the sensing electrode.

The first sensor may be a bioelectrical impedance analysis (BIA) sensor comprising a current source for supplying current to the sensing electrode, wherein the BIA sensor may be configured to obtain an impedance signal of a human body in contact with the sensing electrode.

The first sensor may be a contact-type temperature sensor configured to measure a temperature of a human body in contact with the sensing electrode.

The integrated sensing platform may further include: a light source mounted on the circuit layer to emit light to a reflective area of the sensor cover; and a light receiver mounted on the circuit layer to receive light reflected from the reflective area and to obtain a contact force signal of the measurement target with respect to a measurement target contact area of the sensor cover, wherein, the processor may be further configured to correct the first signal obtained from the first sensor based on the contact force signal obtained from the light receiver along with the temperature signal obtained from the at least one calibration temperature sensor.

The processor may be further configured to adjust the first signal by inputting first signal data obtained from the first sensor, temperature data obtained from the at least one calibration temperature sensor, and contact force data obtained from the light receiver into a first signal correction model.

According to another aspect of the disclosure, there is provided an integrated sensing platform including: a first sensor comprising a light source configured to emit light to a measurement target and a first light receiver configured to receive light reflected from the measurement target; a sensor cover having a portion that allows the light to pass through to the measurement target; an optical splitter configured to distribute the light emitted by the light source to a measurement target contact area and a light intensity measurement area of the sensor cover; a second light receiver configured to measure light intensity information based on intensity of the light propagating to the light intensity measurement area; and a circuit layer electrically connected to the first sensor and the second light receiver and comprising a processor configured to uniformly control the light intensity of the light source based on the light intensity information measured by the second light receiver.

The first sensor may include a photoplethysmogram (PPG) sensor or an optical displacement sensor.

The integrated sensing platform may further include a third light receiver configured to receive the light that is emitted to a reflective area of the sensor cover by the optical splitter and then reflected from the reflective area, and obtain a contact force signal of the measurement target with respect to the measurement target contact area, wherein the circuit layer is configured to correct a first signal obtained from the first light receiver based on contact force signal obtained from the third light receiver.

The processor may be further configured to adjust the first signal by inputting first signal data obtained from the first light receiver and contact force data obtained from the third light receiver into a first signal correction model.

According to another aspect of the disclosure, there is provided an integrated sensing platform package including: a support layer comprising a processor; a first sensor provided on the support layer and configured to obtain a first signal from a measurement target; a second sensor provided on the support layer and configured to obtain a second signal in a vicinity of the first sensor; and wherein the processor is configured to: obtain the first signal from the first sensor; obtain the second signal from the second sensor; and adjust the first signal based on the second signal.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram illustrating a configuration of an integrated sensing platform according to an example embodiment.

FIG. 2 is a graph illustrating an example of a method of correcting a first signal based on a temperature signal.

FIG. 3 is a diagram illustrating an example configuration of the integrated sensing platform shown in FIG. 1 in which a light source is configured as a multi-wavelength light source.

FIG. 4 is a diagram illustrating an example configuration of the integrated sensing platform shown in FIG. 1 in which an element for acquiring a contact force signal is further included.

FIG. 5 is a diagram illustrating an example of a first sensor that acquires information on a measurement target in a non-contact manner.

FIG. 6 is a diagram illustrating a configuration of an integrated sensing platform according to another example embodiment.

FIG. 7 is a diagram illustrating a configuration of an integrated sensing platform according to another example embodiment.

FIG. 8 is a diagram illustrating a configuration of an integrated sensing platform according to another example embodiment.

FIG. 9 is a diagram illustrating another example configuration of a first sensor of FIG. 8.

FIG. 10 is a diagram illustrating still another example configuration of the first sensor of FIG. 8.

FIG. 11 is a diagram illustrating an example configuration of the integrated sensing platform shown in FIG. 8 in which an element for acquiring a contact force signal is further included.

FIG. 12 is a diagram illustrating a configuration of an integrated sensing platform according to another example embodiment.

FIG. 13 is a diagram illustrating an example configuration of the integrated sensing platform shown in FIG. 12 in which an element for acquiring a contact force signal is further included.

FIG. 14 is a diagram illustrating a configuration of an integrated sensing platform according another example embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. Also, descriptions of well-known functions and constructions may be omitted for increased clarity and conciseness.

Details of example embodiments are included in the following detailed description and drawings. Advantages and features of the disclosure, and a method of achieving the same will be more clearly understood from the following embodiments described in detail with reference to the accompanying drawings. Throughout the drawings and the detailed description, unless otherwise described, the same drawing reference numerals will be understood to refer to the same elements, features, and structures.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise.

It will be further understood that when an element is referred to as “comprising” another element, the element is intended not to exclude one or more other elements, but to further include one or more other elements, unless explicitly described to the contrary. In the following description, terms such as “unit” and “module” indicate a unit for processing at least one function or operation, and the “unit” and the “module” may be implemented by using hardware, software, or a combination of hardware and software.

Hereinafter, embodiments of an integrated sensing platform will be described in detail with reference to the accompanying drawings. Various embodiments of the integrated sensing platform may be mounted in a terminal, such as a smartphone, a tablet personal computer (PC), a desktop PC, a notebook PC, a wearable device, and the like.

Here, the wearable device may be implemented as a wristwatch type wearable device, a bracelet-type wearable device, a ring type wearable device, a glasses-type wearable device, an ear-worn type wearable device, or the like. However, the embodiments are not limited to these examples, and the integrated sensing platform may be mounted in hardware manufactured in various forms for use in specialized medical institutions.

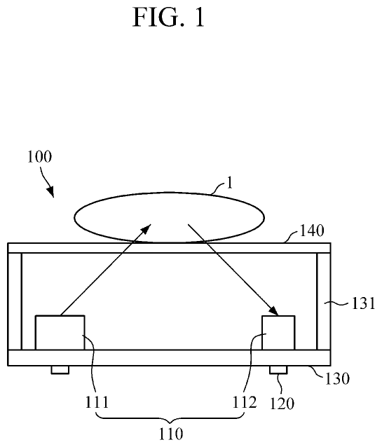

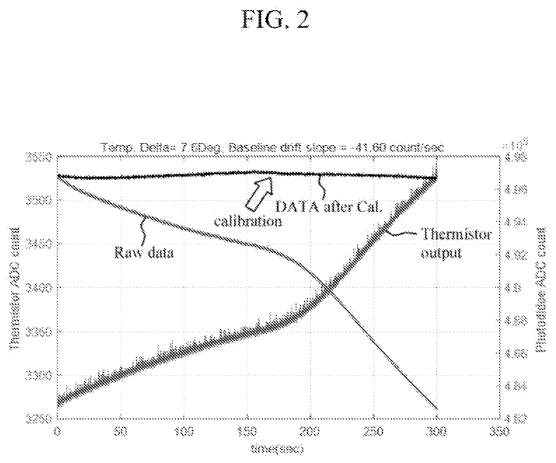

FIG. 1 is a diagram illustrating a configuration of an integrated sensing platform according to an example embodiment. FIG. 2 is a graph illustrating an example of a method of correcting a first signal based on a temperature signal.

Referring to FIGS. 1 and 2, an integrated sensing platform 100 according to the example embodiment may include a first sensor, at least one second sensor, and a circuit layer 130. According to an example embodiment, the first sensor 110 may be a main sensor or a primary sensor configured to acquire a first signal or a primary signal from a measurement target. Here, the “main” or the “primary” may represent the main or the primary biosignal to be measured. According to an example embodiment, the at least one second sensor 120 may be a calibration temperature sensor 120. However, the disclosure is not limited thereto, and as such, the first sensor 110 and the at least one second sensor 120 may include other types of sensors. According to an example embodiment, the circuit layer 130 may, for example, include a processor configured to control a number of hardware or software elements connected to the processor by driving an operating system or application program, and perform various data processing and calculations. Further, the processor may load and process a command or data received from at least one of the other components to a volatile memory and store diverse data in a non-volatile memory.

According to an example embodiment, the first sensor 110 may acquire a first signal corresponding to a measurement target 1. The first sensor 110 may be electrically connected to the circuit layer 130. For example, the first sensor 110 may include a light source 111 and a first light receiver 112. According to an example embodiment, the first light receiver 112 may be a main light receiver.

The light source 111 may be mounted on the circuit layer 130 and may be configured to emit light to the measurement target 1. The light source 111 may include a light emitting diode (LED), a laser diode, a phosphor, or the like. The light source 111 may be driven under the control of the circuit layer 130 to emit light to the measurement target 1.

The first light receiver 112 may be mounted on the circuit layer 130 to receive light reflected from the measurement target 1. The first light receiver 112 may include a photodiode, a photo transistor, a charge-coupled device (CCD), or the like. The first light receiver 112 may provide a received light signal to the circuit layer 130.

The first sensor 110 may be covered by a sensor cover 140 having a portion that transmits light. The sensor cover 140 may transmit the light emitted from the light source 111 through a light-transmitting area, allowing the light to reach the measurement target 1, and may transmit the light reflected from the measurement target 1 through the light-transmitting area, allowing the light to reach the first light receiver 112. According to an example embodiment, the sensor cover 140 may having an opening portion that allows the light to reach the measurement target 1.

According to an example embodiment, a space may be formed between the sensor cover 140 and the circuit layer 130, and the light source 111 and the first light receiver 112 may be accommodated and protected in the space. According to an example embodiment, the integrated sensing platform 100 may include side portions 131 or sidewalls between the sensor cover 140 and the circuit layer 130. According to an example embodiment, the light source 111 and the first light receiver 112 may be spatially separated by a package material between the sensor cover 140 and the circuit layer 130, so that the light emitted from the light 111 can be prevented from directly entering into the first light receiver 112.

The first sensor 110 may be a photoplethysmogram (PPG) sensor. In this case, as shown in FIG. 1, the first sensor 110 may acquire a PPG signal from a body part, such as a finger, in contact with a contact surface of the sensor cover 140.

The PPG signal may be used in measuring biometric information, such as heart rate, oxygen saturation, blood pressure, etc. of a human body. The PPG signal obtained by the first sensor 110 may be provided to the circuit layer 130, and the circuit layer 130 may estimate biometric information by applying an estimation model defining a correlation between the PPG signal and biometric information. The estimation model may be defined through various methods such as a linear function, linear/nonlinear regression analysis, and the like.

The calibration temperature sensor 120 may acquire a temperature signal corresponding to a temperature in a vicinity of the first sensor 110. According to an example embodiment, the calibration temperature sensor 120 may acquire a temperature of an area around the first sensor 110 or near the first sensor 110. The calibration temperature sensor 120 may be electrically connected to the circuit layer 130. The calibration temperature sensor 120 may include a thermistor and the like.

The calibration temperature sensor 120 may be mounted on the circuit layer 130. According to an example embodiment, two temperature sensors 120 may be provided. For example, a first temperature sensor 120 may be provided at an area corresponding to the light source 111 and a second temperature sensor 120 may be provided at an area corresponding to the first light receiver 112 to measure a temperature. However, the disclosure is not limited to two temperature sensors, and as such, according another example embodiment, one or three or more temperature sensors 120 may be provided to measure a temperature around the light source 111 and the first light receiver 112.

According to an example embodiment, the calibration temperature sensor 120 may be provided at a position where it can accurately measure the temperature around the light source 111 and the first light receiver 112 through heat flux analysis. When a plurality of temperature sensors 120 are provided to measure the temperature around the first sensor 110, an average value of the measured temperature values may be used.

According to an example embodiment, the circuit layer may include one or more electrical components connected via circuitry. The circuit layer 130 may be heated by current passing therethrough. That is, the circuit layer 130 may be heated by Joule heating by which heat is generated when current passes through a conductor. In addition, the circuit layer 130 may be heated by the operation of the light source 111 constituting the first sensor 110. In addition, when the circuit layer 130 includes components, such as an analog front end (AFE) through which high current passes, a low-dropout regulator (LDO), and the like, the circuit layer 130 may be heated by these components.

The characteristics of the light source 111 may vary according to a change in ambient temperature due to heating of the circuit layer 130 or a change in temperature due to self-heating. The characteristics of the first light receiver 112 may also vary according to a change in ambient temperature due to heating of the circuit layer 130. Accordingly, a first signal obtained from the first light receiver 112 may be affected by a change in characteristics of the light source 111 and the first light receiver 112 due to the change in temperature.

The first signal obtained from the first light receiver 112 is a measurement value based on light intensity, the first signal may include an alternating current (AC) component and a direct current (DC) component. In the first signal, drift of the DC component over time may occur due to changes in temperature characteristics of the light source 111 and the first light receiver 112. The drift of the DC component may deteriorate the measurement accuracy when various physical quantities are measured using light.

When the DC component of the first signal is used as a main measurement signal, a correction needs to be made to minimize drift of the DC component for accurate measurement. To this end, the calibration temperature sensor 120 may monitor the changes in temperature characteristics of the light source 111 and the first light receiver 112 by detecting the temperature around the light source 111 and the first light source 112, thereby increasing the measurement accuracy by a correction of the first signal.

The circuit layer 130 may be electrically connected to the first sensor 110 and the calibration temperature sensor 120. The circuit layer 130 may control the light source 111 of the first sensor 110 to be driven. The circuit layer 130 may receive the first signal from the first light receiver 112 of the first sensor 110 and process the first signal. The circuit layer 130 may receive the temperature signal from the calibration temperature sensor 120 and process the temperature signal.

The circuit layer 130 may correct the first signal obtained from the first sensor 110 on the basis of the temperature signal obtained from the calibration temperature sensor 120. The circuit layer 130 may include a processor or a controller configured to process various signals and transmit the processed signals.

The circuit layer 130 may correct the first signal by inputting first signal data obtained from the first sensor 110 and temperature data obtained from the calibration temperature sensor 120 into a first signal correction model. The first signal correction model may be obtained as a relational expression between the first signal data and the temperature data through a pre-calibration process. The first signal correction model may be obtained through various methods such as a linear function, linear/nonlinear regression analysis, and the like.

For example, as shown in FIG. 2, when the first light receiver 112 of the first sensor 110 includes a photodiode and the calibration temperature sensor 120 includes a thermistor, raw data obtained from the photodiode and a thermistor output value obtained from the thermistor may be input into the first signal correction model to correct the raw data. Therefore, the raw data may be corrected to minimize the drift of the DC component, so that the measurement accuracy of various physical quantities can be increased.

The integrated sensing platform 100 described above may include a sensor unit including the first sensor 110 and the calibration temperature sensor 120, and the circuit layer 130. According to an example embodiment, the circuit layer 130 may be an integrated circuit (IC) chip and/or may include a digital signal processor (DSP), a microprocessor, a central processing unit (CPU), a micro controller unit (MCU), a micro processing unit (MPU), a controller, an application processor (AP), a communication processor (CP), an ARM processor, a microprocessor, a graphics processing unit (GPU), an artificial intelligence (AI) processor, a neural processing unit (NPU) or other processing circuitry. The sensor unit and the circuit layer 130 may be configured as separate components and assembled as a set, or may be stacked and configured in a single packaging form, such as a system on chip (SoC), a system in package (SiP), large scale integration (LSI) with a built-in processing algorithm, or as an application specific integrated circuit (ASIC) or field programmable gate array (FPGA) type, or the like.

The sensor unit and the circuit layer 130 may be stacked by a semiconductor process. The calibration temperature sensor 120 may be included in the circuit layer 130. According to an example embodiment, the calibration temperature sensor 120 may be provided on the circuit layer 130. However, the disclosure is not limited thereto, and as such, according to another example embodiment, the calibration temperature sensor 120 may be embedded in the circuit layer 130. When the sensor unit and the circuit layer 130 are configured in a single packaging form, it may be beneficial in terms of measurement accuracy, and it may have advantages in mass production and yield improvement since component size can be reduced and the manufacturing process can be simplified.

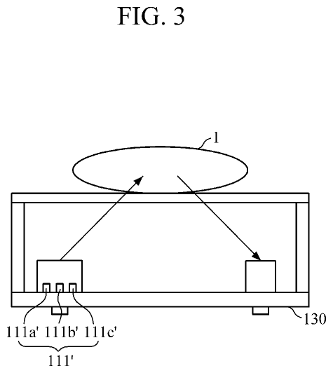

FIG. 3 is a diagram illustrating an example configuration of the integrated sensing platform shown in FIG. 1 in which a light source is configured as a multi-wavelength light source.

The light source 111′ may be a multi-wavelength light source. For example, the multi-wavelength light source 111′ may include a first light source 111a′ configured to emit infrared light, a second light source 111b′ configured to emit red light, and a third light source 111c′ configured to emit green light. When the measurement target 1 is a body part, infrared light and red light may be used to measure oxygen saturation according to a difference in light absorption of oxygenated and non-oxygenated hemoglobin within red blood cells. Green light may be used to measure a pulse wave. The circuit layer 130 may selectively control the first, second, and third light sources 111a′, 111b′, and 111c′ to be driven according to the measurement purpose.

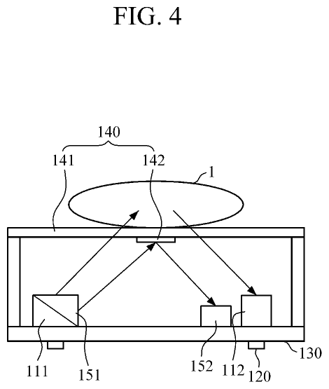

FIG. 4 is a diagram illustrating an example configuration of the integrated sensing platform shown in FIG. 1 in which an element for acquiring a contact force signal is further included.

Referring to FIG. 4, the integrated sensing platform may further include an optical splitter 151 and a second light receiver 152. According to an example embodiment, the second light receiver 152 may be a sub-light receiver. The optical splitter 151 may distribute the light emitted by the light source 111 to a measurement target contact area 141 and a reflective area 142 of the sensor cover 140.

The measurement target contact area 141 is an area where the measurement target 1 is in contact with the sensor cover 140 and the force of the measurement target 1 is applied. A portion of the measurement target contact area 141 may transmit light.

The reflective area 142 is an area where the light distributed by the optical splitter 151 is reflected from the sensor cover 140. The reflective area 142 may be formed by coating a reflective material onto the inner surface of the sensor cover 140 or by attaching a reflective member, such as a mirror, onto the inner surface of the sensor cover 140. The inner surface of the sensor cover 140 may be made of a material having reflective properties to form the reflective area 142.

The optical splitter 151 may distribute light intensity, emitted by the light source 111, at a predetermined ratio using a mirror, a prism, or the like to the measurement target contact area 141 and the reflective area 142 of the sensor cover 140 along first and second light paths. The light traveling along the first light path may be reflected from the measurement target 1 in contact with the measurement target contact area 141 and be received by the first light receiver 112. The light traveling along the second light path may be reflected from the reflective area 142 and be received by the second light receiver 152. The positional relationship between the light source 111 and the first light receiver 112 and the second light receiver 152 may be specified in order to efficiently receive light through the first and second light paths.

The second light receiver 152 may be mounted on the circuit layer 130 to receive the light reflected from the reflective area 142 and acquire a contact force signal of the measurement target 1 with respect to the measurement target contact area 141. The second light receiver 152 may include a photodiode, a photo transistor, a CCD, or the like. The second light receiver 152 may provide the received light signal to the circuit layer 130.

When the measurement target 1 makes contact with the measurement target contact area 141 and applies force to the measurement target contact area 141, the integrated sensing platform may deform. Accordingly, a light path may change between the contact surface of the measurement target 1 and the mounted surface of the first sensor 110 on the circuit layer. The light path change may include contact force information of the measurement target 1. The second light receiver 152 may detect the light path change so that the contact force information of the measurement target 1 can be acquired.

The first signal obtained from the first light receiver 112 may be affected by the contact force of the measurement target 1. A change in contact force of the measurement target 1 may cause drift of the DC component over time in the first signal. The second light receiver 152 may detect the contract force information of the measurement target 1 according to the light path change, thereby increasing the measurement accuracy by a correction of the first signal.

The circuit layer 130 may correct the first signal obtained from the first light receiver 112 on the basis of the contact force signal obtained from the second light receiver 152 together with the temperature signal obtained from the calibration temperature sensor 120.

The circuit layer 130 may input the first signal data obtained from the first light receiver 112, the temperature data obtained from the calibration temperature sensor 120, and the contact force data obtained from the second light receiver 152 into the first signal correction model to correct the first signal.

The first signal correction model may be obtained as a relational expression between temperature data for the first signal data and the contact force data through a pre-calibration process. The first signal correction model may be obtained through various methods, such as multiple regression analysis.

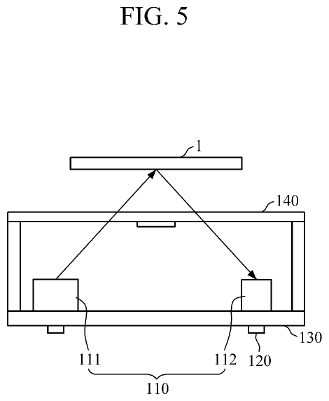

FIG. 5 is a diagram illustrating an example of a first sensor that acquires information on a measurement target in a non-contact manner.

Referring to FIG. 5, the first sensor 110 may be an optical displacement sensor. The first sensor 110 may use the first light receiver 112 to receive light that is emitted from the light source 111 to the measurement target through a light-transmitting area of the sensor cover 140 and reflected from the measurement target 1. In this case, the light intensity received by the first light receiver 112 may vary according to a distance between the first sensor 110 and the measurement target 1. Thus, the light signal received by the first light receiver 112 may be used to measure a displacement of the measurement target 1.

The light signal obtained by the first light receiver 112 may be provided to the circuit layer 130 and the circuit layer 130 may calculate displacement information by using a calculation model defining a correlation between the light signal and the displacement information. The calculation model may be defined through various methods such as a linear function, linear/nonlinear regression analysis, and the like. The circuit layer 130 may correct a displacement information signal obtained from the first light receiver 112 on the basis of the temperature signal obtained from the calibration temperature sensor 120, thereby increasing the accuracy of displacement measurement.

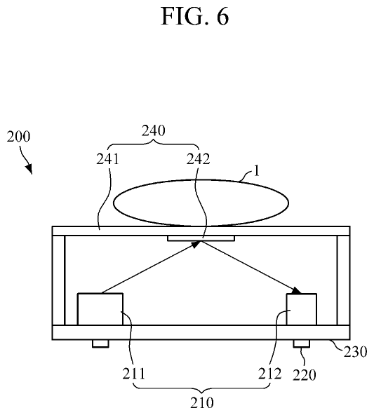

FIG. 6 is a diagram illustrating a configuration of an integrated sensing platform according to another example embodiment.

Referring to FIG. 6, in an integrated sensing platform 200 according to the example embodiment, a first sensor 210 may be an optical force sensor. The first sensor 210 may include a light source 211 and a first light receiver 212.

The light source 211 may be mounted on a circuit layer 230 to emit light to a reflective area 242 of a sensor cover 240 pressed by the force of a measurement target 1. The light source 211 may include an LED, a laser diode, a phosphor, or the like.

The entire area of the sensor cover 240 may not transmit light. The sensor cover 240 may have a measurement target contact area 241. The measurement target contact area 241 is an area where the measurement target 1 is in contact with the sensor cover 240 and the force of the measurement target 1 is applied.

The reflective area 242 may be formed by coating a reflective material onto the inner surface of the sensor cover 240 or by attaching a reflective member, such as a mirror, onto the inner surface of the sensor cover 240. The inner surface of the sensor cover 240 may be made of a material having reflective properties to form the reflective area 242.

The first light receiver 212 may be mounted on the circuit layer 230 to receive light reflected from the reflective area 242 of the sensor cover 240. The first light receiver 212 may include a photodiode, a photo transistor, a CCD, or the like. Light intensity received by the first light receiver 212 may vary according to an amount of deformation of the sensor cover 240 pressed by a force applied by the measurement target 1. Therefore, the light signal received by the first light receiver 212 may be used to measure a pressing force of the measurement target 1.

The light signal obtained by the first light receiver 212 may be provided to the circuit layer 230 and the circuit layer 230 may calculate force information by using a calculation model defining a correlation between the light signal and force information. The calculation model may be defined through various methods such as a linear function, linear/nonlinear regression analysis, and the like. The circuit layer 230 may correct a force information signal obtained from the first light receiver 212 on the basis of the temperature signal obtained from the calibration temperature sensor 220, thereby increasing the accuracy of force measurement. The calibration temperature sensor 220 may be configured to have the same structure as the calibration temperature sensor 120 of the example described above.

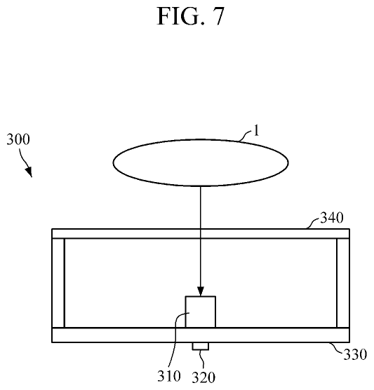

FIG. 7 is a diagram illustrating a configuration of an integrated sensing platform according to another example embodiment.

Referring to FIG. 7, in an integrated sensing platform 330 according to the example embodiment, a first sensor 310 may include a light receiver mounted on a circuit layer 330 to received light radiated from a measurement target 1. The light receiver may include a photodiode, a photo transistor, a CCD, or the like. The light receiver may provide the received light signal to the circuit layer 330.

The first sensor 310 may be covered by a sensor cover 340 having a portion that transmits light. For example, the first sensor 310 may be an optical temperature sensor. The light receiver may detect light intensity that changes with the temperature of a human body.

The light receiver may receive light in infrared wavelengths that is radiated from the human body. The light signal obtained by the light receiver may be provided to the circuit layer 330, and may be applied to a calculation model defining a correlation between the light signal and temperature information to calculate temperature information. The calculation model may be defined through various methods such as a linear function, linear/nonlinear regression analysis, and the like. The circuit layer 330 may correct a body temperature signal obtained from the light receiver of the first sensor 310 on the basis of the temperature signal obtained from the calibration temperature sensor 320, thereby increasing the accuracy of body temperature measurement.

In another example, the first sensor 310 may be a light intensity sensor. The light receiver may measure an amount of ambient light around the measurement target 1 and provide the measured amount of ambient light to the circuit layer 330. The circuit layer 330 may correct a light amount signal obtained from the light receiver of the first sensor 310 on the basis of the temperature signal obtained from the calibration temperature sensor 320, thereby increasing the accuracy of light amount measurement. The calibration temperature sensor 320 may be configured to have the same structure as the calibration temperature sensor 120 of the example described above.

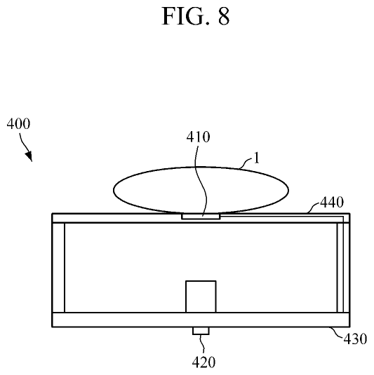

FIG. 8 is a diagram illustrating a configuration of an integrated sensing platform according to another example embodiment.

Referring to FIG. 8, in an integrated sensing platform 400 according to the example embodiment, a first sensor 410 may include a sensing electrode. The sensing electrode may be electrically connected to a circuit layer 430, while being disposed on an outer surface of a sensor cover 440 that covers the circuit layer 430. The sensing electrode may be mounted to be exposed to the outer surface of the sensor cover 440. The entire area of the sensor cover 440 may not transmit light.

The first sensor 410 may be an ECG sensor that acquires an electrocardiogram signal of a human body in contact with the sensing electrode. When measuring the electrocardiogram, the sensing electrode may be in contact with a finger of the body part and detect a fine electrical signal from the skin of the finger for each heart beat. At a resting phase, each cardiac muscle cell has a negative charge. These negative charges are decreased due to the inflow of cations, and thus the depolarization occurs and the heart contracts. During each heartbeat, the heart provides an orderly depolarization wave form spreading out from the signal coming out from a sinoatrial node to whole ventricle.

The electrical signal detected by the sensing electrode may be provided to the circuit layer 430. The circuit layer 430 may measure the electrocardiogram by analyzing the electrical signal detected by the sensing electrode. The circuit layer 430 may measure the electrocardiogram by using an estimation model defining a correlation between the electrical signal and the electrocardiogram. The estimation model may be defined through various methods such as a linear function, linear/nonlinear regression analysis, and the like.

The circuit layer 430 may correct an electrocardiogram information signal obtained from the first sensor 410 on the basis of the temperature signal obtained from the calibration temperature sensor 420, thereby increasing the accuracy of electrocardiogram measurement. The calibration temperature sensor 420 may be configured to have the same structure as the calibration temperature sensor 120 of the example described above.

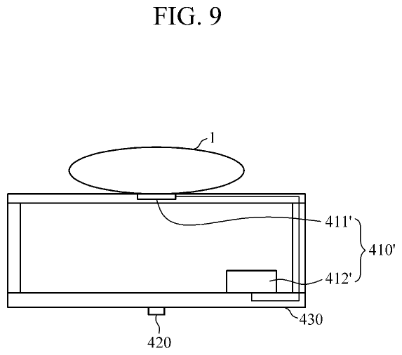

FIG. 9 is a diagram illustrating another example configuration of a first sensor of FIG. 8.

Referring to FIG. 9, the first sensor 410′ may be a bioelectrical impedance analysis (BIA) sensor that includes a current source 412′ for supplying current to a sensing electrodes 411′ and obtains an impedance signal of the human body in contact with the sensing electrodes 411′.

When measuring bioelectrical impedance, the sensing electrode 411′ applies a current signal to a finger of the body part in contact with the sensing electrode 411′ and detect a voltage signal from the finger. The circuit layer 430 may control the current source 412′ to apply current to the sensing electrode 411′, thereby applying a current signal to the finger, and may measure bioelectrical impedance by analyzing the voltage signal detected by the sensing electrode 411′.

The circuit layer 430 may estimate biometric information, such as a ratio of bio-tissue like body fat, based on the measured bioelectrical impedance. The circuit layer 430 may estimate the biometric information by using an estimation model defining a correlation between the measured bioelectrical impedance and the biometric information. The estimation model may be defined through various methods such as a linear function, linear/nonlinear regression analysis, and the like.

The circuit layer 430 may correct an impedance signal obtained from the first sensor 410′ on the basis of the temperature signal obtained from the calibration temperature sensor 420, thereby increasing the accuracy of impedance measurement.

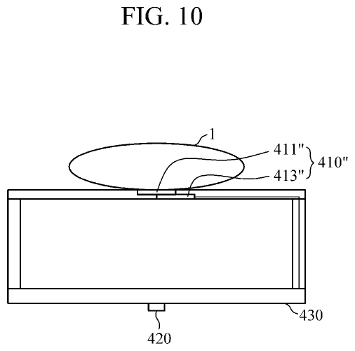

FIG. 10 is a diagram illustrating still another example configuration of the first sensor of FIG. 8.

Referring to FIG. 10, a first sensor 410″ may be a contact-type temperature sensor that measures the temperature of a human body in contact with a sensing electrode 411′. The first sensor 410″ may include a temperature sensing element 413″ and measure the temperature of a body part in contact with the sensing electrode. The temperature sensing element 413″ may include a thermocouple, a thermistor, or the like.

An electrical signal output from the first sensor 410″ may be provided to the circuit layer 430. The circuit layer 430 may calculate temperature information by using a calculation model defining a correlation between the electrical signal and temperature information. The calculation model may be defined through various methods such as a linear function, linear/nonlinear regression analysis, and the like.

The circuit layer 430 may correct a temperature information signal obtained from the first sensor 410″ on the basis of the temperature signal obtained from the calibration temperature sensor 420, thereby increasing the accuracy of temperature measurement.

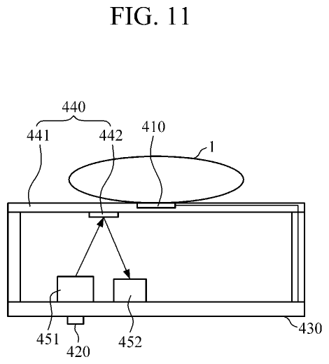

FIG. 11 is a diagram illustrating an example configuration of the integrated sensing platform shown in FIG. 8 in which an element for acquiring a contact force signal is further included.

Referring to FIG. 11, an integrated sensing platform may further include a light source 451 and a light receiver 452. The light source 451 may be mounted on a circuit layer 430 to emit light to a reflective area 442 of a sensor cover 440. The light source 451 may include an LED, a laser diode, a phosphor, or the like.

The light receiver 452 may be mounted on the circuit layer 430 to receive the light reflected from the reflective area 442 and acquire a contact force signal of a measurement target 1 with respect to a measurement target contact area 441 of the sensor cover 440. The light receiver 452 may include a photodiode, a photo transistor, a CCD, or the like. The light receiver 452 may provide the received light signal to the circuit layer 430.

The light receiver 452 may detect contact force information of the measurement target 1, as illustrated in FIG. 4, thereby increasing measurement accuracy by a correction of the first signal. The circuit layer 430 may correct the first signal obtained from the first sensor 410 on the basis of a contact force signal obtained from the light receiver 452 together with the temperature signal obtained from the calibration temperature sensor 420.

The circuit layer 430 may correct the first signal by inputting first signal data obtained from the first sensor 410, temperature data obtained from the calibration temperature sensor 420, and contact force data obtained from the light receiver 452 into a first signal correction model. In this embodiment, the integrated sensing platform may include the first sensor 410′ illustrated in FIG. 9, or the first sensor 410″ illustrated in FIG. 10.

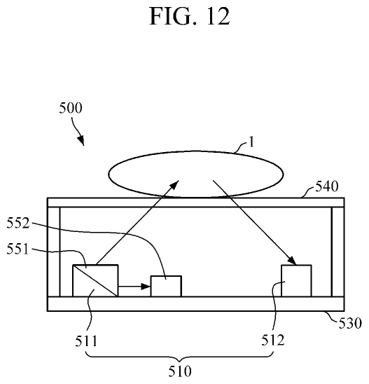

FIG. 12 is a diagram illustrating a configuration of an integrated sensing platform according to another example embodiment.

Referring to FIG. 12, an integrated sensing platform 500 according to the example embodiment may include a first sensor 510, a circuit layer 530, an optical splitter 551, and a second light receiver 552.

The first sensor 510 may use a light source 511 to emit light to a measurement target 1 and may use a first light receiver 512 to receive the light reflected from the measurement target 1. The first sensor 510 may be covered by a sensor cover 540 having a portion that transmits light.

The light source 511 and the first light receiver 512 may be mounted on the circuit layer 530. The light source 511 may include an LED, a laser diode, a phosphor, or the like. The first light receiver 512 may be configured as a device that is robust to temperature changes, such as a silicon photodiode, but is not limited thereto. The first sensor 510 may be either a PPG sensor or an optical displacement sensor.

The optical splitter 551 may distribute the light emitted by the light source 511 to a measurement target contact area and a light intensity measurement area of the sensor cover 540. The optical splitter 551 may distribute light intensity, emitted by the light source 511, at a predetermined ratio using a mirror, a prism, or the like to the measurement target contact area and the light intensity measurement area of the sensor cover 540 along first and second light paths.

The light traveling along the first light path may be reflected from the measurement target 1 in contact with the measurement target contact area and be received by the first light receiver 512. The light traveling along the second light path may be directly received by the second light receiver 552.

The second light receiver 552 may measure light intensity propagating to the light intensity measurement area. The second light receiver 552 may be configured as a silicon photodiode, but is not limited thereto. The second light receiver 552 may function to directly and accurately monitor the light intensity emitted by the light source 511.

The circuit layer 530 may be electrically connected to the first sensor 510 and the second light receiver 552. The circuit layer 530 may estimate biometric information by using an estimation model defining a correlation between a PPG signal obtained by the first sensor 510 including a PPG sensor and the biometric information. The circuit layer 530 may calculate displacement information by using a calculation model defining a correlation between a light signal obtained by the first sensor 510 including an optical displacement sensor and the displacement information.

The circuit layer 530 may uniformly control the light intensity of the light source 511 on the basis of the light intensity information measured by the second light receiver 552. The output of the light source 511 may vary according to various factors, such as a change in ambient temperature due to heating of the circuit layer 530 or a change in temperature due to self-heating. Accordingly, a first signal obtained from the first light receiver 512 may be affected by a change in output of the light source 511. The circuit layer 530 may uniformly control the light intensity of the light source 511, thereby allowing the accurate measurement of the first signal obtained from the first light receiver 512.

The circuit layer 530 may receive a light intensity signal from the second light receiver 552 and uniformly control the light intensity of the light source 511 to a target value in real time. The circuit layer 530 may receive the light intensity signal from the second light receiver 552 and uniformly control the light intensity of the light source 511 on the basis of a linear model.

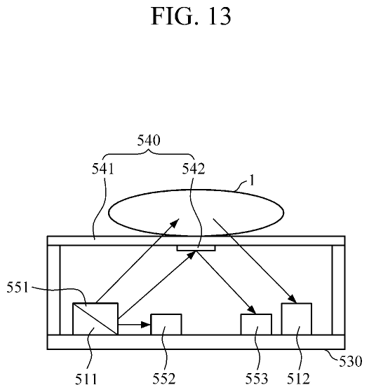

FIG. 13 is a diagram illustrating an example configuration of the integrated sensing platform shown in FIG. 12 in which an element for acquiring a contact force signal is further included.

Referring to FIG. 13, the integrated sensing platform may further include a third light receiver 553. According to an example embodiment, the third light receiver 153 may be another sub-light receiver. The third light receiver 553 may receive the light that is emitted from the light source 511 to the reflective area 542 of the sensor cover 540 through the optical splitter 551 and then reflected from the reflective area 542, and may obtain a contact force signal of the measurement target 1 with respect to the measurement target contact area 541. The third light receiver 553 may be configured as a silicon photodiode, but is not limited thereto. The third light receiver 553 may provide the received light signal to the circuit layer 530.

The third light receiver 553 may detect contact force information of the measurement target 1, as illustrated in FIG. 4, thereby increasing measurement accuracy by a correction of the first signal. The circuit layer 530 may correct a first signal obtained from the first light receiver 512 on the basis of the contact force information obtained from the third light receiver 553.

The circuit layer 530 may correct the first signal by inputting first signal data obtained from the first light receiver 512 and contact force data obtained from the third light receiver 553 into a first signal correction model. The first signal correction model may be obtained as a relational expression between the first signal data and the contact force data through a pre-calibration process. The first signal correction model may be obtained through various methods such as a linear function, linear/nonlinear regression analysis, and the like.

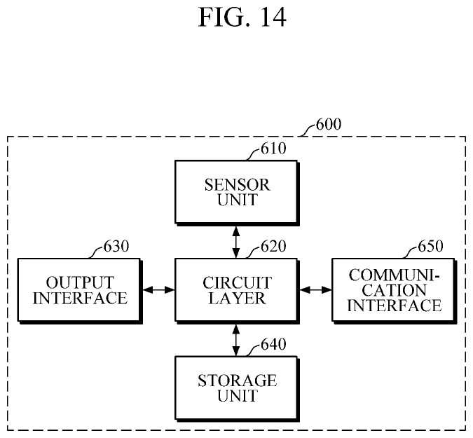

FIG. 14 is a diagram illustrating a configuration of an integrated sensing platform according to another embodiment.

Referring to FIG. 14, an integrated sensing platform 600 according to the example embodiment may include a sensor 610, a controller circuit layer 620, an output interface 630, a storage 640, and a communication interface 650.

The sensor 610 may include one or more of the sensors in the above-described integrated sensing platforms 100, 200, 300, 400, and 500 according to an example embodiment. The circuit layer 620 may correspond to the circuit layers in the above-described integrated sensing platforms 100, 200, 300, 400, and 500 according to an example embodiment. The configuration of the sensor unit 610 and the circuit layer 620 is described above, and thus the detailed description thereof will not be reiterated.

The output interface 630 may output a processing process or a processing result of the sensor unit 610 and/or the circuit layer 620. The output interface 630 may provide information to a user using visual, auditory, and tactile methods, such as a visual output module (e.g., a display), an audio output module (e.g., a speaker), a haptic module, and the like.

The storage unit 640 may store various data necessary for the sensor unit 610 and/or the circuit layer 620 and/or the processing results. The storage unit 640 may include at least one type of storage medium, such as a flash memory type, a hard disk type, a multimedia card micro type, a card type memory (e.g., secure digital (SD) or extreme digital (XD) memory), random access memory (RAM), static random access memory (SRAM), read-only memory (ROM), electrically erasable programmable read-only memory (EEPROM), programmable read-only memory (PROM), a magnetic memory, a magnetic disk, and an optical disk.

The communication interface 650 may communicate with an external device to transmit and receive various data necessary for the sensor unit 610 and/or the circuit layer 620 and/or the processing results to and from the external device. Here, the external device may be medical equipment, a printer to print out results, or a display to display the results. In addition, the external device may be a digital TV, a desktop computer, a cellular phone, a smartphone, a tablet PC, a laptop computer, a personal digital assistant (PDA), a portable multimedia player (PMP), a navigation system, an MP3 player, a digital camera, a wearable device, and the like, but is not limited thereto.

The communication interface 650 may communicate with the external device by using various communication techniques such as Bluetooth communication, Bluetooth Low Energy (BLE) communication, Near Field Communication (NFC), WLAN communication, Zigbee communication, Infrared Data Association (IrDA) communication, wireless fidelity (Wi-Fi) Direct (WFD) communication, Ultra-Wideband (UWB) communication, Ant+ communication, Wi-Fi communication, Radio Frequency Identification (RFID) communication, 3G communication, 4G communication, 5G communication, and the like. However, these are merely examples, and the present disclosure is not limited thereto.

Although various example embodiments have been described, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

1. An integrated sensing platform comprising:
a first sensor configured to obtain a first signal from a measurement target;
at least one calibration temperature sensor configured to obtain a temperature signal in a vicinity of the first sensor; and
a circuit layer electrically connected to the first sensor and the at least one calibration temperature sensor and comprising a processor configured to:
obtain the first signal from the first sensor;
obtain the temperature signal from the at least one calibration temperature sensor; and
adjust the first signal based on the temperature signal.

2. The integrated sensing platform of claim 1, wherein the processor is further configured to adjust the first signal by inputting first signal data obtained from the first sensor and temperature data obtained from the at least one calibration temperature sensor into a first signal correction model.

3. The integrated sensing platform of claim 1, wherein the first sensor comprises a light source mounted on the circuit layer and configured to emit light to the measurement target and a first light receiver mounted on the circuit layer and configured to receive light reflected from the measurement target,
wherein the integrated sensing platform further comprises a sensor cover having a portion that allows the light to pass through to the measurement target.

4. The integrated sensing platform of claim 3, wherein the first sensor comprises one of a photoplethysmogram (PPG) sensor or an optical displacement sensor.

5. The integrated sensing platform of claim 3, wherein the light source is a multi-wavelength light source.

6. The integrated sensing platform of claim 3, further comprising:
an optical splitter configured to distribute the light emitted by the light source to a measurement target contact area and a reflective area of the sensor cover; and
a second light receiver mounted on the circuit layer and configured to receive light reflected from the reflective area and obtain a contact force signal of the measurement target with respect to the measurement target contact area,
wherein the processor is further configured to adjust the first signal obtained from the first light receiver based on the contact force signal obtained from the second light receiver along with the temperature signal obtained from the at least one calibration temperature sensor.

7. The integrated sensing platform of claim 6, wherein the processor is further configured to adjust the first signal by inputting first signal data obtained from the first light receiver, temperature data obtained from the at least one calibration temperature sensor, and contact force data obtained from the second light receiver into a first signal correction model.

8. The integrated sensing platform of claim 1, wherein the first sensor is an optical force sensor that comprises a light source mounted on the circuit layer to emit light to a reflective area of a sensor cover pressed by a force of the measurement target and a first light receiver mounted on the circuit layer to receive light reflected from the reflective area of the sensor cover.

9. The integrated sensing platform of claim 1, wherein the first sensor comprises a light receiver mounted on the circuit layer and configured to receive light radiated from the measurement target, and
wherein the integrated sensing platform further comprises a sensor cover having a portion that allows the light to pass from to the measurement target.

10. The integrated sensing platform of claim 9, wherein the first sensor comprises an optical temperature sensor or a light intensity sensor.

11. The integrated sensing platform of claim 1, wherein the first sensor comprises a sensing electrode that is electrically connected to the circuit layer, and
wherein the first sensor is provided on an outer surface of a sensor cover covering the circuit layer.

12. The integrated sensing platform of claim 11, wherein the first sensor is an electrocardiogram (ECG) sensor configured to acquire an electrocardiogram signal of a human body in contact with the sensing electrode.

13. The integrated sensing platform of claim 11, wherein the first sensor is a bioelectrical impedance analysis (BIA) sensor comprising a current source for supplying current to the sensing electrode,
wherein the BIA sensor is configured to obtain an impedance signal of a human body in contact with the sensing electrode.

14. The integrated sensing platform of claim 11, wherein the first sensor is a contact-type temperature sensor configured to measure a temperature of a human body in contact with the sensing electrode.

15. The integrated sensing platform of claim 11, further comprising:
a light source mounted on the circuit layer to emit light to a reflective area of the sensor cover; and
a light receiver mounted on the circuit layer to receive light reflected from the reflective area and to obtain a contact force signal of the measurement target with respect to a measurement target contact area of the sensor cover,
wherein, the processor is further configured to correct the first signal obtained from the first sensor based on the contact force signal obtained from the light receiver along with the temperature signal obtained from the at least one calibration temperature sensor.

16. The integrated sensing platform of claim 15, wherein the processor is further configured to adjust the first signal by inputting first signal data obtained from the first sensor, temperature data obtained from the at least one calibration temperature sensor, and contact force data obtained from the light receiver into a first signal correction model.

17. An integrated sensing platform comprising:
a first sensor comprising a light source configured to emit light to a measurement target and a first light receiver configured to receive light reflected from the measurement target;
a sensor cover having a portion that allows the light to pass through to the measurement target; an optical splitter configured to distribute the light emitted by the light source to a measurement target contact area and a light intensity measurement area of the sensor cover;
a second light receiver configured to measure light intensity information based on intensity of the light propagating to the light intensity measurement area; and
a circuit layer electrically connected to the first sensor and the second light receiver and comprising a processor configured to uniformly control the light intensity of the light source based on the light intensity information measured by the second light receiver.

18. The integrated sensing platform of claim 17, wherein the first sensor comprises a photoplethysmogram (PPG) sensor or an optical displacement sensor.

19. The integrated sensing platform of claim 17, further comprising:
a third light receiver configured to receive the light that is emitted to a reflective area of the sensor cover by the optical splitter and then reflected from the reflective area, and obtain a contact force signal of the measurement target with respect to the measurement target contact area,
wherein the circuit layer is configured to correct a first signal obtained from the first light receiver based on contact force signal obtained from the third light receiver.

20. The integrated sensing platform of claim 19, wherein the processor is further configured to adjust the first signal by inputting first signal data obtained from the first light receiver and contact force data obtained from the third light receiver into a first signal correction model.

21. An integrated sensing platform package comprising:
a support layer comprising a processor;
a first sensor provided on the support layer and configured to obtain a first signal from a measurement target;
a second sensor provided on the support layer and configured to obtain a second signal in a vicinity of the first sensor; and
wherein the processor is configured to:
obtain the first signal from the first sensor;
obtain the second signal from the second sensor; and
adjust the first signal based on the second signal.
